(19) United States
(12) Patent Application Publication
(10) Pub. No.: US 2013/0304112 A1
(43) Pub. Date: Nov. 14, 2013

(54) APPARATUS AND METHOD FOR ALTERING THE ARTERIAL PULSE WAVEFORM OF A BODY

(71) Applicants: Choon Meng Ting, New Century (SG); Wei Nee Serene Chang, Loyang Gardens (SG)

(72) Inventors: Choon Meng Ting, New Century (SG); Wei Nee Serene Chang, Loyang Gardens (SG)

(73) Assignee: HEALTHSTATS INTERNATIONAL PTE LTD., Singapore (SG)

(21) Appl. No.: 13/980,831

(22) PCT Filed: Nov. 9, 2011
(86) PCT No.: PCT/SG11/00398

§ 371 Date: Jul. 19, 2013

(30) Foreign Application Priority Data
Jan. 21, 2011 (SG) 201100445-4

(51) Int. Cl. A61B 17/132

(52) U.S. Cl. CPC A61B 17/1325
USPC 606/203

(57) ABSTRACT

An apparatus and method for altering reflected arterial pulse of a body is disclosed. The apparatus comprises a belt having a compression member arranged to apply a pressure to the body, the belt being arranged to be positioned such that the compression member applies the pressure to one of at least two pressure points between the xiphis sternum and navel of the body, the belt adjustable to provide at least two pressure levels to each of the at least two pressure points and a blood pressure monitoring device arranged to obtain a beat-to-beat arterial pulse waveform when the pressure is applied at each of the pressure points and at each of the at least two pressure levels; and analyze the waveform to determine the optimum position for the compression device to apply the pressure to the body to alter the pulse waveform.

### FIELD OF INVENTION

The invention relates to an apparatus and method for altering the arterial pulse waveform of a body. In particular but not exclusively, the invention relates to a non-invasive apparatus and method for altering arterial pulse waveform.

### BACKGROUND TO THE INVENTION

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

Arterial pulse waveform provides information of blood pressure generated by the heart when it relaxes and contracts. The contraction of the heart generates a pulse wave which travels along the arterial walls of the arterial tree of a person. Generally, the waveform comprises two main components, a forward moving wave and a reflected wave. The forward wave is generated when the heart (ventricles) contracts during systole. This wave travels down the aorta from the heart and gets reflected at the bifurcation or the “cross-road” of the aorta into 2 iliac vessels. In a normal healthy person, the reflected wave usually returns in the diastolic phase, after the closure of the aorta valves. The returned wave produces a ‘notch’ in the pulse waveform which helps in the perfusion of the heart through the blood vessels as it pushes the blood through the blood vessels.

Various performance indices observable from arterial pulse waveform provides an indication of the cardiac health of a person. Such performance indices include upstroke gradient which indicates ventricular mechanical function; area under the pulse waveform curve which is the overall cardiac load; and dicrotic pressure which provides information on the pressure for perfusion of coronary arteries. In addition, the reflected (returned wave) measures arterial wall stiffness. For example, in a person suffering from cardiovascular diseases, or for an aged person, the arteries are stiffer than normal. As a result, the velocity at which the reflected waveform returns is relatively faster than in a normal healthy person and may enter into the systolic phase earlier than normal, i.e. before the aorta valves closes. The resultant final blood pressure reading is increased. This is undesirable as it increase the after-load of the heart, requiring the heart to pump harder to overcome the reflected pulse wave.

Medication (invasive drugs) may be used to improve one or more of the above indices in order reduce the risk of a heart attack, or to treat cardiovascular diseases. However, medication may bring about it certain side effects to the person which is also undesirable.

Non-invasive intervention methods for regulating the distribution of blood around the body have gained popularity in the recent years as an alternative to the invasive medication. In particular, external counter-pulsation (ECP) and enhanced external counter-pulsation (EECP) apparatus and methods have become prevalent. A typical external counter-pulsation apparatus comprises multiple cuffs wrapped around the four limbs of a patient. In some ECP systems, additional cuffs may be wrapped around the hip region and/or buttocks of the patient. Pressure is then applied sequentially from the distal to the proximal portion of each limb. Controller(s) are used to control the onset of inflation and deflation of each cuff. The controller is synchronized with the patient's electrocardiogram (ECG). The cuffs are timed to inflate at the beginning of diastole and deflate at the beginning of systole based on the ECG. During the inflation portion of the cycle, the calf cuffs inflate first, then the lower thigh cuffs and finally the upper thigh cuffs. The cuffs are generally inflated to about 300 mmHg.

When the controller is synchronized correctly with the ECG, the ECP system works to decrease the after load that the heart has to pump against, and increase the preload that fills the heart, thereby increasing the cardiac output.

However, it is important that the timing of deflating and inflating the cuffs is precise and exact in order to achieve the desired results. An error in the synchronization of operation of the cuffs may be detrimental to the patient and in the worst case, fatalities may result. Also, the relatively high pressure ‘squeezing’ on the patient's thighs and calves during the process produce considerable amount of discomfort to the patient. Bruises on the skin of the thighs arising from ECP treatments are not uncommon.

In addition, the ECP/EECP apparatus is not portable and as a consequence, is deployed in a clinic or hospital which can accommodate this type of apparatus.

It is therefore desirable to provide an apparatus and method for altering the reflected arterial pulse of a body to alleviate the above problems.

### SUMMARY OF THE INVENTION

Throughout this document, unless otherwise indicated to the contrary, the terms “comprising”, “consisting of”, and the like, are to be construed as non-exhaustive, or in other words, as meaning “including, but not limited to”.

In a first aspect of the present invention, there is provided an apparatus for altering the arterial pulse waveform of a body comprising: a belt having a compression member arranged to apply a pressure to the body, the belt being arranged to be positioned such that the compression member applies the pressure to one of at least two pressure points between the xiphis sternum and navel of the body, the compression member adapted to provide at each of the at least two pressure points at least two pressure levels; and

a blood pressure monitoring device arranged to: obtain a beat-to-beat arterial pulse waveform when the pressure is applied at each of the pressure points and at each of the at least two pressure levels; and analyze the waveform to determine the optimum position for the compression device to apply the pressure to the body to alter the pulse waveform.

The apparatus provides for a passive method of altering arterial pulse waveform without the need for synchronization with various phases of heart beat cycle.

Preferably, the beat-to-beat arterial pulse waveform is obtained from a radial artery. Advantageously, the beat-to-beat blood pressure monitoring device is non-invasive. More advantageously, the arterial pulse waveform analyzer is integrated with the beat-to-beat blood pressure monitoring device.

It is envisaged that the at least two pressure points between the xiphis sternum and navel of the body are located at approximately at the ⅓ and ⅔ of the distance between the xiphis sternum and navel. Further, it is preferred that the pressure levels are varied according to the adjustment of the belt to at least two diameters around the body.

Alternatively, instead of adjusting the belt to at least two diameters around the body, the belt may comprise a knob for adjusting the pressure level applied to each of the at least two pressure points. It is envisaged that the compression member may be adjustable to at least five different tensions of pressure.

In accordance with a second aspect of the present invention there is a method for altering the arterial pulse waveform of a body comprising the steps of: locating the xiphis sternum and navel of the body; determining the distance between the xiphis sternum and navel; selecting a pressure point on the distance; and applying pressure to the pressure point for a predetermined period.

Preferably, the applied pressure is substantially constant throughout the predetermined period. Preferably, the pressure point is selected from at least two pressure points located on the distance between the xiphis sternum and navel. It is envisaged that the at least two pressure points are located at approximately at the ⅓ and ⅔ of the distance between the xiphis sternum and navel.

Preferably, the pressure point and amount of pressure exerted at the pressure point are determined based on the body's response to at least one arterial waveform indices.

The arterial waveform indices may be selected from one or more of the following: Systolic peak time (SPT); Systolic upstroke gradient (SUG); Augmentation produced by reflected arterial wave; Net dicrotic notch to peak height; and Pulse rate (PR).

### BRIEF DESCRIPTION OF THE DRAWING

In the figures, which illustrate, by way of example only, embodiments of the present invention, in which:—

FIG. 1 shows the perspective view of the apparatus for altering the arterial pulse waveform of a body according to the embodiment of the invention.

FIG. 2a shows the perspective view of the compression belt; FIG. 2b shows another perspective view of the compression belt; and FIG. 2c shows the compression belt in use.

FIG. 3a to c illustrates the various arterial waveform performance indices.

FIG. 4 is the flowchart for obtaining the baseline arterial pulse waveform of a body 100.

FIG. 5 illustrates the method of obtaining and marking the relevant positions on the abdomen according to step 34 of FIG. 4. It also shows the general location of the Xiphis sternum and navel on a body.

FIGS. 6 and 7 are the flowcharts for obtaining waveform pulse for a body at different tensions at positions P₁ and P₂ respectively.

FIG. 8 is the flowchart of the selection process for an optimal set of tension and position for the apparatus to be used for a particular body 100.

FIG. 9 illustrates the compression of the aorta when the apparatus is used in the correct way.

FIG. 10 is a table of a typical matrix database of a patient 100 according to the embodiment of the invention.

FIG. 11 is the table of a typical matrix database of another patient 100 according to the embodiment of the invention.

FIG. 12 is the table of an alternative matrix database of a patient 100 using two tension levels instead of three.

FIGS. 13a and 13b show examples of valid and invalid waveform data collected by the arterial waveform analyzer 16.

FIG. 14 shows an example of how normalized/average arterial pulse waveform of a patient 100 is obtained;

FIGS. 15a and 15b illustrate an alternative compression belt and compression member according to another embodiment of the invention;

FIG. 16 illustrates a position locator for obtaining the relevant positions on the abdomen in lieu of the method of FIG. 5, and

FIGS. 17a to 17c illustrates further results based the alternative compression belt.

Other arrangements of the invention are possible and, consequently, the accompanying drawings are not to be understood as superseding the generality of the preceding description of the invention.

### DETAILED DESCRIPTION OF THE EMBODIMENT

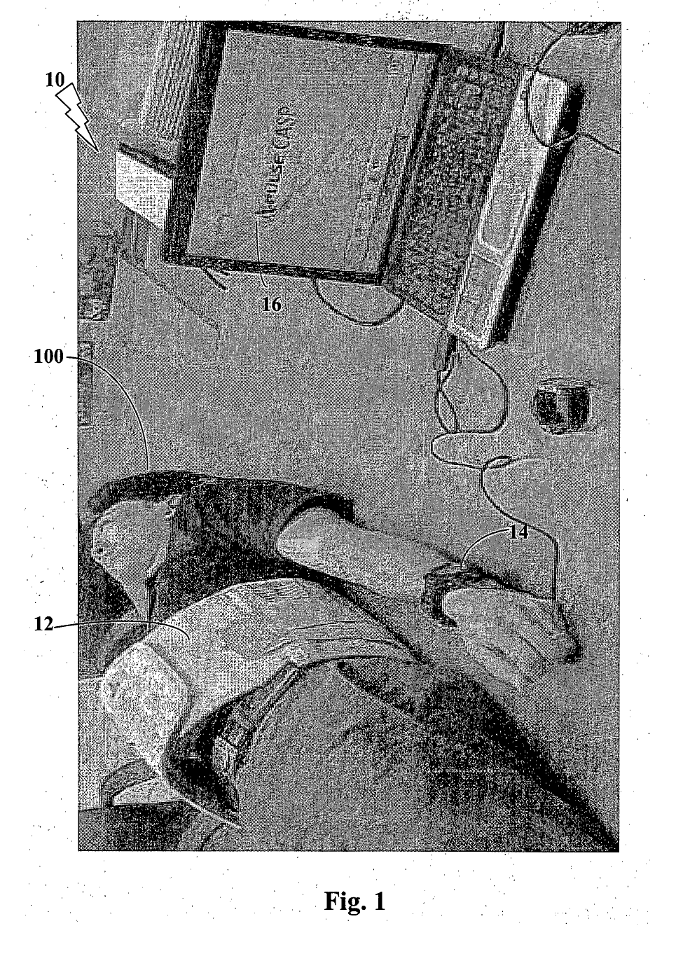

In accordance with an embodiment of the invention there is provided an apparatus 10 for altering the arterial pulse waveform of a body. The apparatus 10 comprises a compression belt 12, a blood pressure monitoring device 14 and an arterial pulse waveform analyser 16 as shown in FIG. 1.

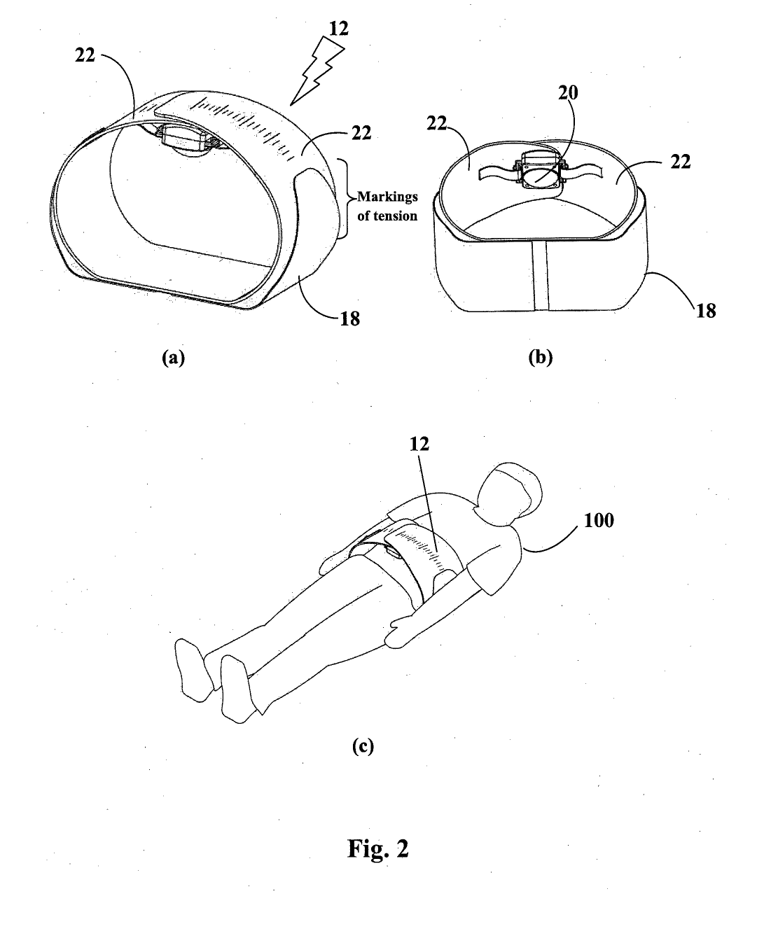

The compression belt 12 comprises an elastic band 18 and a compression member 20. As illustrated in FIG. 2, the elastic band 18 is typically an elastic band with a fastening means 22. Fastening means 22 is typically a double strap 22. The elastic band 18 is similar to the waist/back supports available in the market and known to a skilled person. In addition, the tightness of the band when wrapped around a body 100 is adjustable to varying degrees of tension—minimum, moderate, and maximum. In the preferred embodiment, the tightness of the band when wrapped around a body 100 is adjustable to three degrees of tension—minimum, moderate, and maximum. The positions on the elastic band 18 corresponding to the three different pressure levels providing the three degrees of tension are conveniently marked.

Compression member 20 is a hemispherical shaped protrusion. Compression member 20 is suitably a plunger located on one of the double strap 22 of the elastic band 18 such that when the double strap 22 are attached to each other for fastening, the elastic band 18 wraps around the patient 100. In use, the compression member 20 contacts a pressure point (subsequently elaborated) on the abdominal region of the body 100 and exerts a pressure on the abdomen based on the tightness of the band 16. It is to be appreciated that the compression belt 10, when worn around the body 100 and appropriately fastened, exerts a pressure based via the compression member 20 on the abdomen without constricting the overall circumference of the abdomen.

The blood pressure monitoring device 14 is typically a non-invasive blood pressure monitoring device. Blood pressure monitoring device 14 is capable of obtaining arterial blood pressure pulse waveform data on a beat-to-beat basis. The blood pressure monitoring device 14 is in data communication with the arterial pulse waveform analyzer 16. The arterial pulse waveform analyzer 16 is capable of obtaining arterial pulse waveform data from the blood pressure monitoring device 14 and performs pulse waveform analysis to obtain arterial pulse waveform indices. The arterial waveform indices comprise:

Systolic peak time (SPT)—to be minimized;
Systolic upstroke gradient (SUG)—to be maximized;
Augmentation produced by reflected arterial wave—to be minimized;
Net dicrotic notch to peak height—to be maximized, and
Pulse rate (PR)—to be minimized.

In the embodiment, the blood pressure monitoring device 14 is the BPro device of HealthStats International Pte Ltd. The arterial pulse waveform analyzer 16 is the A-Pulse CASP application software installed on a computer (laptop or otherwise) via one or more computer readable media. The BPro device is worn around the wrist of the patient 100 and obtains beat-to-beat blood pressure readings of a radial artery at the wrist region of the patient 100. The blood pressure monitoring device 14 and the arterial pulse waveform analyzer 16 may be integrated into one single device, i.e. the A-Pulse CASP application software may be incorporated in the BPro device. Such integration provides for greater portability and convenience. Prior to use, the apparatus is customized for a patient 100 as follows. It is to be appreciated that the customization process has to be performed for each different patient 100 or for the same patient 100 if certain time period, such as 2 weeks has elapsed since the previous customization.

Details of the patient 100 are first obtained. The details include information such as the age, weight and height of the patient 100. The details are recorded (Step 30). In addition, at least three resting blood pressure readings of the patient 100 are taken over a period of about 15-20 minutes. It is to be appreciated that the resting blood pressure readings are obtained with an interval of 3-5 minutes between each reading using the blood pressure monitoring device 14. The blood pressure readings are then averaged. The averaged blood pressure reading and the corresponding arterial pulse waveforms forms the calibration blood pressure readings for subsequent customization and analysis as elaborated below.

The patient 100 is then made to lie down in a supine position. Care must be taken to ensure that the patient is maintaining a regular breathing pattern (Step 32).

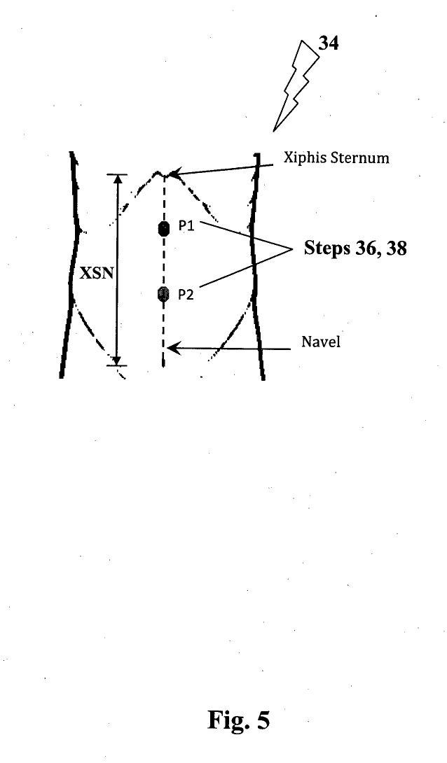

The Xiphis sternum and navel of the patient 100 are located and marked. The distance XSN between the Xiphis sternum and navel positions is calculated (Step 34)—see also FIG. 5.

The distance XSN is divided into three approximately equal segments (step 36) with two pressure points P₁ and P₂ dividing the three segments marked out (step 38).

The calibrated blood pressure values are input in the blood pressure monitoring device 14 (step 40).

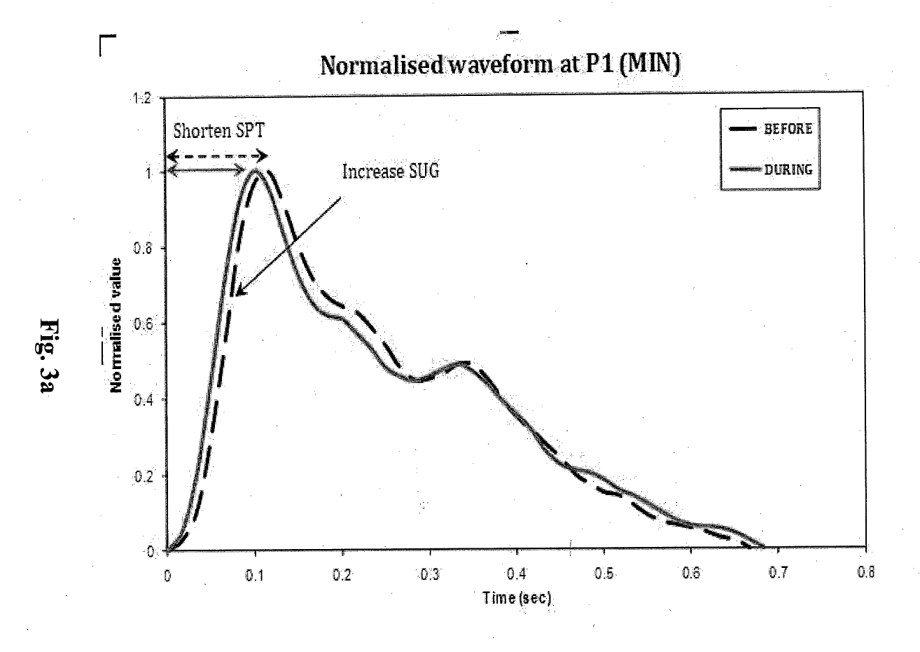

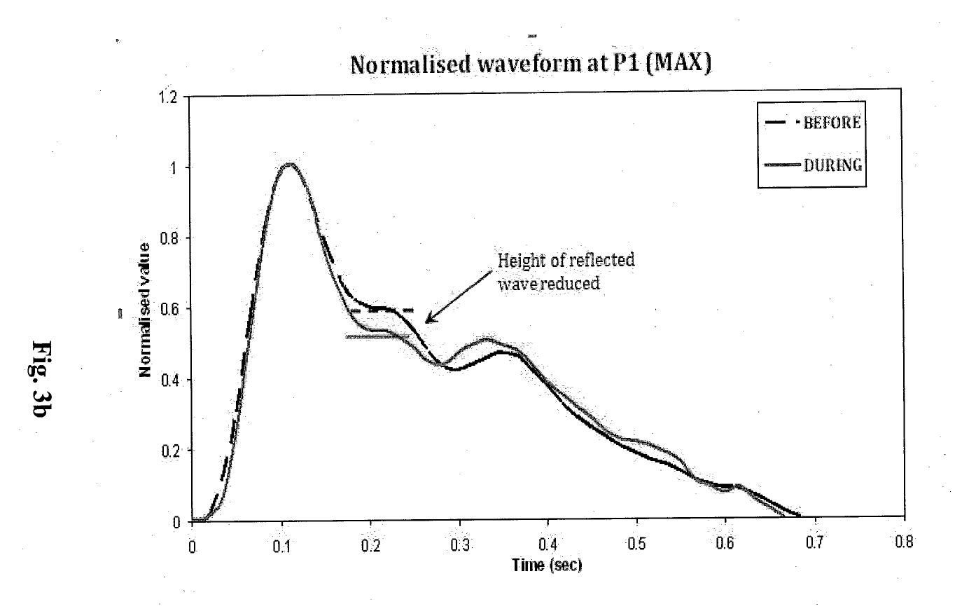

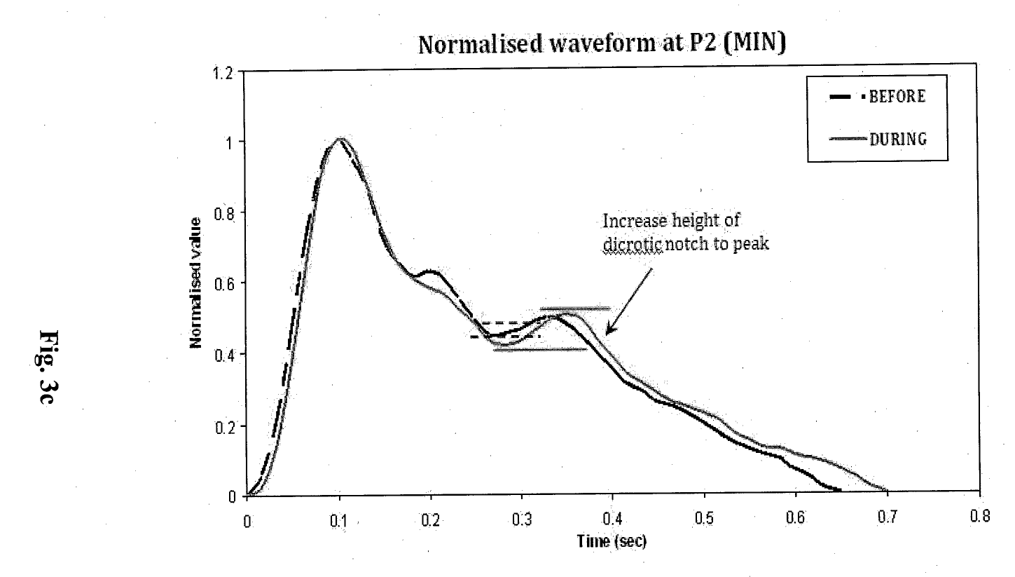

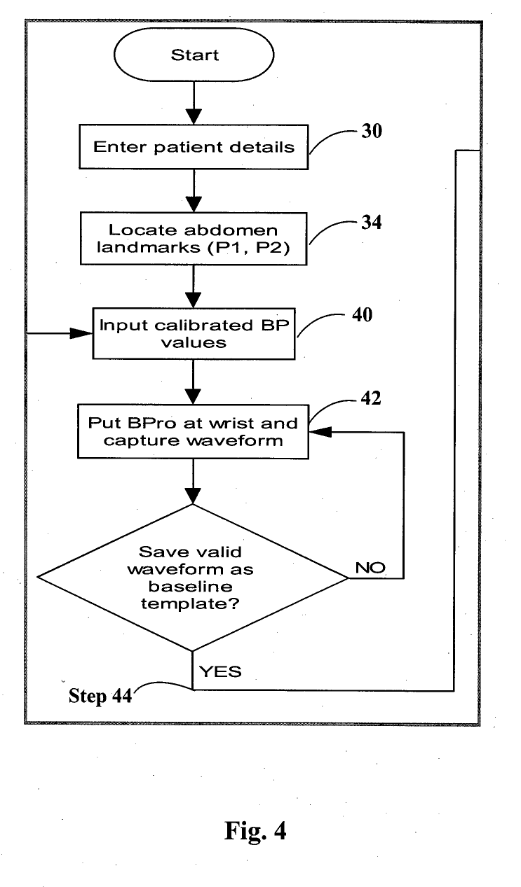

The blood pressure monitoring device 14 is attached to the patient 100 (Step 42). Blood pressure monitoring device 14 is configured to obtain the resting beat-to-beat radial arterial pulse waveform. The arterial pulse waveform analyzer 16, installed as software in a laptop, is in data communication with the blood pressure monitoring device 14. The waveform analyzer 16 calculates the following indices for baselineing purposes from the arterial pulse waveform (see FIG. 3).

(a.) Systolic peak time (SPT)—shown in FIG. 3a,
(b.) Systolic upstroke gradient (SUG)—shown in FIG. 3a,
(c.) Augmentation produced by reflected arterial wave—shown in FIG. 3b,
(d.) Net dicrotic notch to peak height—shown in FIG. 3c, and
(e.) pulse rate (PR)—not shown.

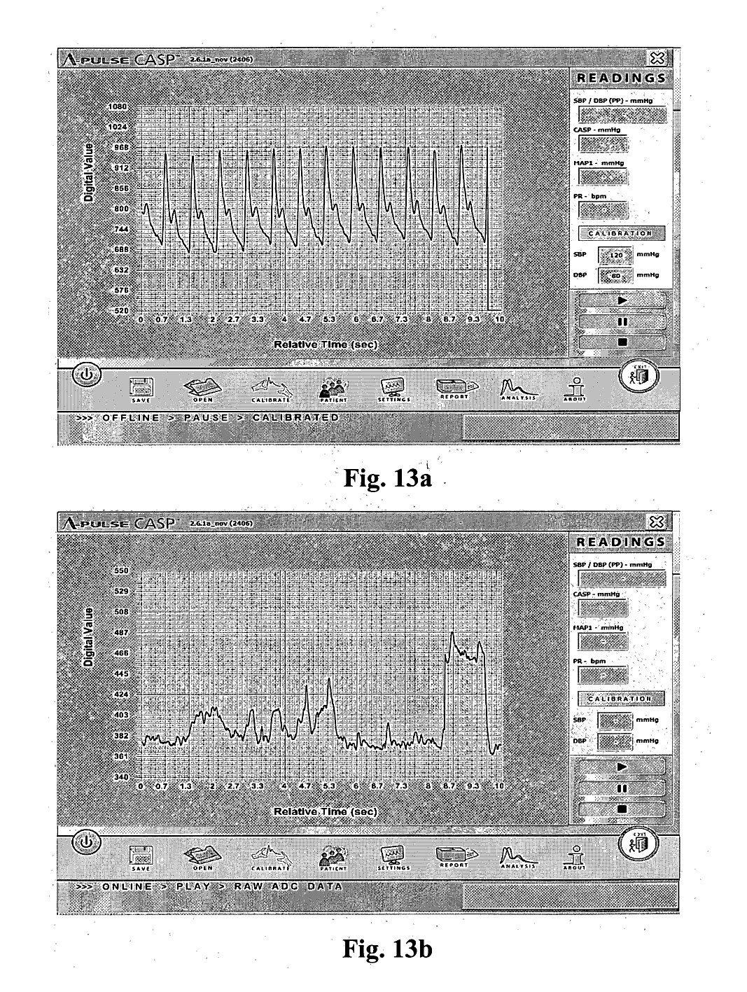

A check is performed to ensure that the arterial pulse waveform obtained is valid (i.e. within acceptable range and limits). Step 42 is repeated if the arterial pulse waveform obtained is invalid. FIG. 13a shows an example of a valid waveform data collected by the arterial pulse waveform analyzer 16. It could be seen that the waveform data is generally repetitive over the various time cycles. Conversely, FIG. 13b shows an example of an invalid waveform data collected by the arterial pulse waveform analyzer 16. Such waveform is generally highly irregular and is characterized by sudden spikes or depression.

Once the arterial pulse waveform is determined to be valid, the waveform is stored as a baseline template (Step 44).

The compression belt 12 is next worn around the patient 100 such that the compression member 20 is adjusted to contact the pressure point P₁ (Step 50). The double strap 22 is adjusted to the two diameters around the sides of the abdomen of the body so as to produce a constant pressure level T₁ at pressure point P₁ which corresponds to the minimum tension level (Step 52). The blood pressure monitoring device 14 is then configured to obtain the beat-to-beat radial arterial pulse waveform during the compression (Step 54). The arterial pulse waveform is saved as position P₁, T₁ (step 56). The matrix database is updated accordingly (Step 58).

Step 52 to 58 is repeated at pressure point P₁ for tension level T₂ (moderate) and T₃(maximum).

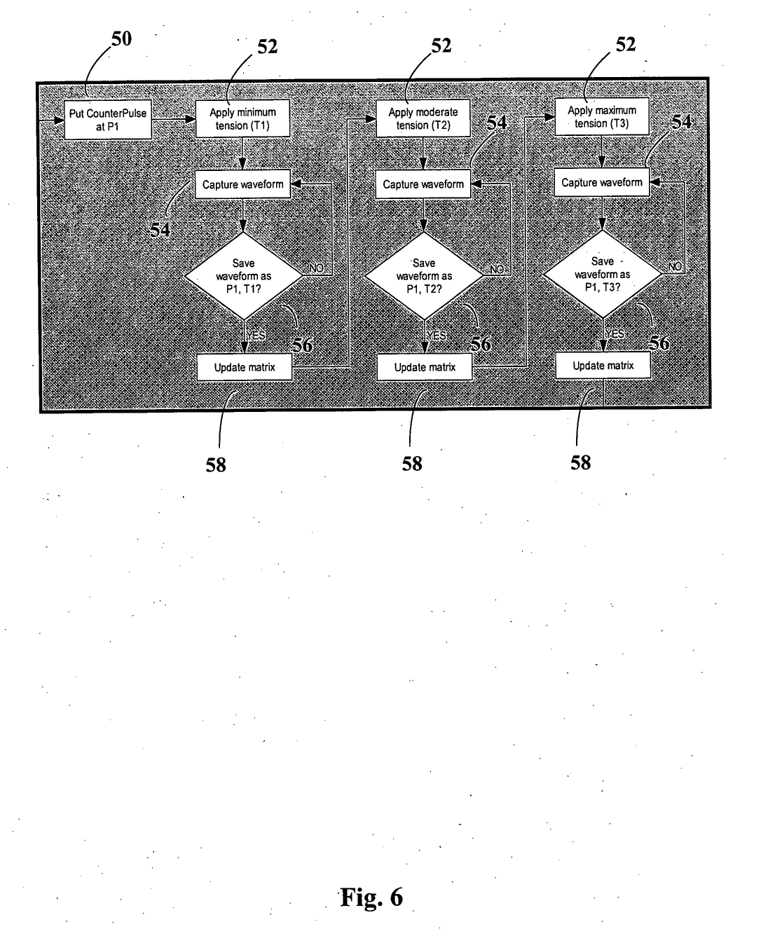

The compression belt 12 is adjusted such that the compression member 20 exerts a pressure at the pressure point P₂ (see FIG. 6).

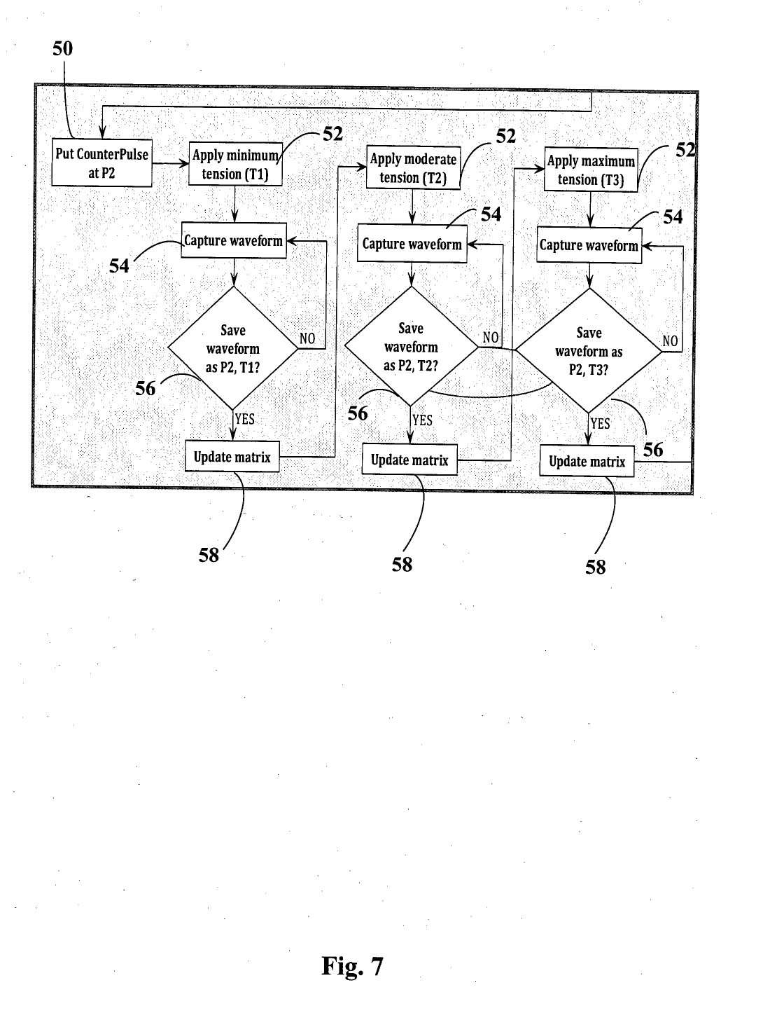

Step 52 to 58 is repeated for tension level T₁, T₂ and T₃ respectively for pressure point P₂ (See FIG. 7).

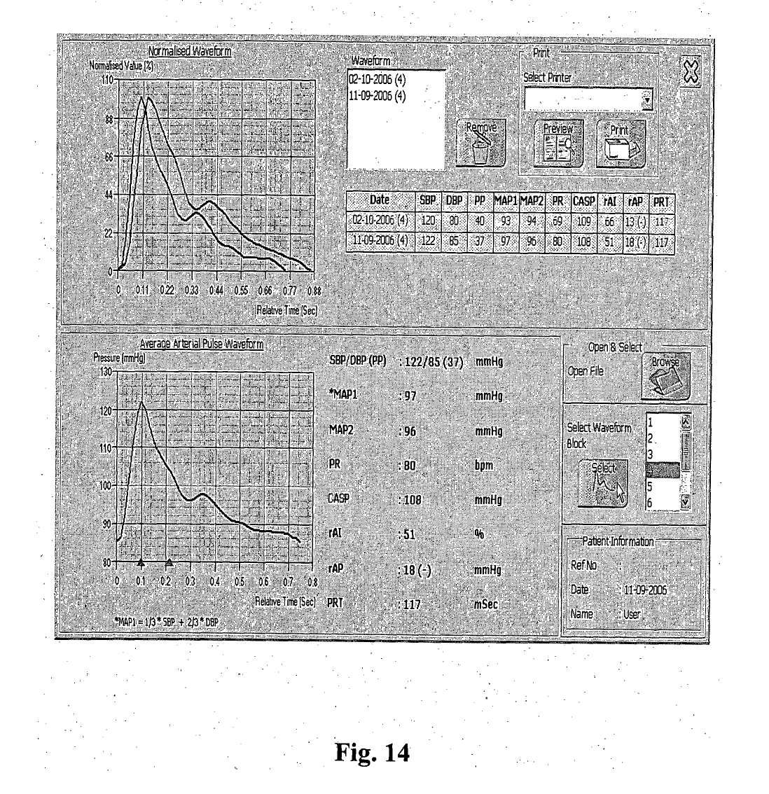

A total of six arterial pulse waveforms and one baseline arterial pulse waveform are collected from the patient 100. The collected data are then normalized with respect to the calibrated blood pressure values. Normalization is performed by the arterial pulse waveform analyzer 16. In the context of the embodiment, the normalized pulse waveform and the average pulse waveform are similar except that the average waveform uses mmHg as the unit of measure while the normalized waveform uses percentage (%), where 100% corresponds to the maximum amplitude of the waveform; for the y-axis (see FIG. 14). The normalized waveforms are superimposed for a best fit when compared to the same baseline. FIG. 14 shows the superimposed waveforms obtained by the arterial pulse waveform analyzer 16 for normalization.

The analyzer 16 then calculates the five indices as mentioned above (step 60):

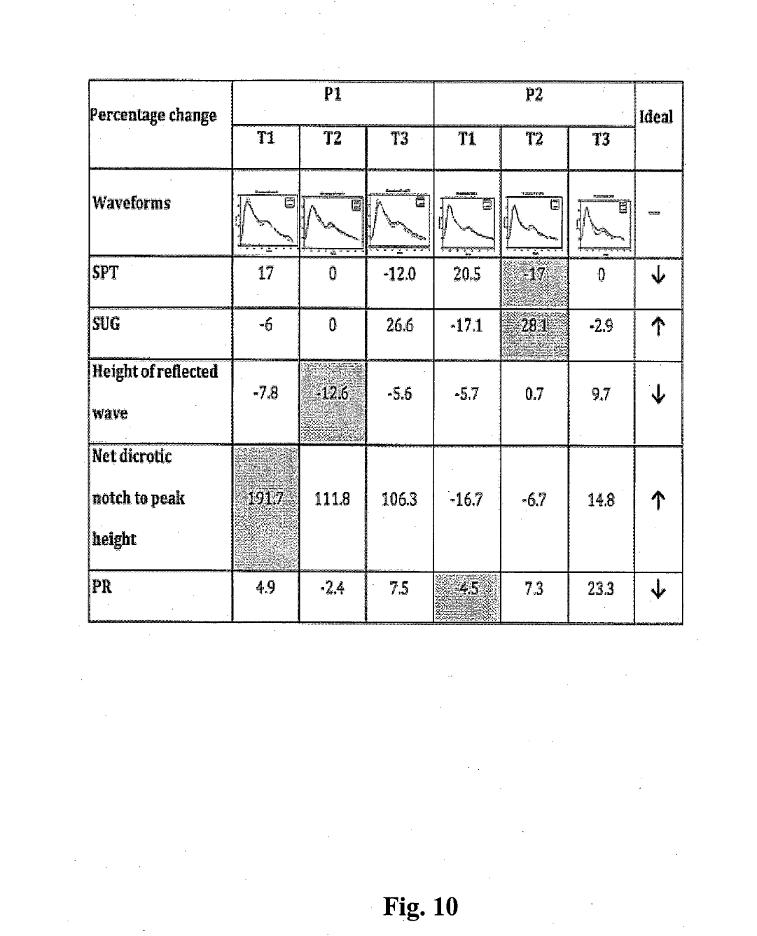

Each normalized arterial pulse waveform is compared with the baseline template waveform as obtained in step 44. The percentage (%) change of each of the five indices is calculated and the database is updated. An example of the completed database matrix is shown in FIG. 10.

FIG. 10 presents each of the five indices corresponding to each combination of pressure points {P₁ or P₂} and tension {T₁; T₂; or T₃} in a typical matrix database of a particular patient 100 after the arterial pulse waveform readings are obtained and the five indices computed. For each of the six arterial pulse waveforms obtained at different pressure points, tension combination; i.e. (P₁, T₁); (P₁, T₂); (P₁, T₃); (P₂, T₁); (P₂, T₂) and (P₂, T₃); thirty indices values (% change relative to baseline pulse waveform) are obtained.

From the obtained indices, the optimum position and tension are selected based on the any one of the following five objectives as desired by the patient 100, ideally under the direction/advice of a qualified medical practitioner.

Minimize systolic peak time (↓ SPT);
Maximize systolic upstroke gradient (↑ SUG);
Minimize/Eliminate augmentation produced by reflected wave (↓ height of reflected wave);
Maximize net dicrotic notch to peak height; and
Minimize pulse rate (↓ PR)

For example, if the patient 100 is required to optimize his SPT or SUG, the P₂, T₂ combination will be selected as the optimum combination as the SPT of the patient has decreased by 17% during the wearing of the compression belt 10 as compared to the baseline; and the SUG has increased by 28.1%.

In summary, the cells corresponding to the best improvement for each combination of (position, tension) are highlighted (Step 62).

### From FIG. 10:

the {P₂, T₂} combination provides the best improvement in SPT and SUG, since the SPT of the patient has decreased by 17% during the wearing of the compression belt 10 as compared to the baseline; and the SUG has increased by 28.1%.
the {P₁, T₂} combination provides the best improvement in terms of decreasing the reflected arterial wave pulse, as the height of the reflected pulse has decreased by 12.6%;
the {P₁, T₁} combination provides the best improvement in terms of increasing the Net dicrotic notch to peak height (increased by 191.7%);
the {P₂, T₁} combination provides the best improvement in terms of decreasing the pulse rate (decreased by 4.5%)

If it is discovered there are two or more combination of {P_{X}, T_{Y}} which provides the same optimal result (Step 64), the analyzer 14 checks if a lower tension is available from the two or more combination (Step 66). If so, the optimal combination with the lower tension is selected.

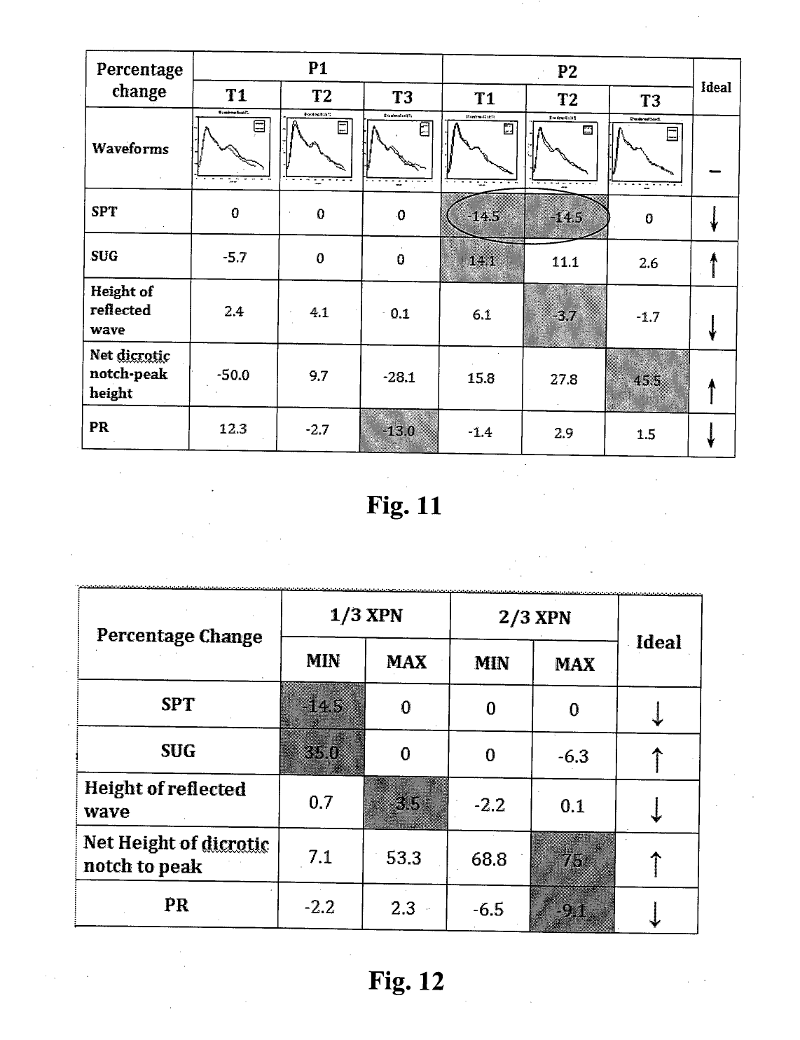

For example, FIG. 11 shows a typical matrix database of another patient 100. From the matrix database, the combination {P₂, T₁}; and {P₂, T₂} produces the same level of improvement in terms of 14.5% decrease of SPT. In such a situation, the lower tension T₁ will be selected as the optimum combination as it causes relatively lower level of discomfort to the patient 100.

Along the same line of reducing discomfort, if the two or more combinations produce the same level of improvement at same tension but different pressure positions, the lower pressure positions P₂ will be used as it causes relatively lower level of discomfort to the patient 100 (Step 68).

Upon determining the optimal {position, tension} combination, the apparatus 10 is ready for use according to the needs of the patient 100 (Step 70).

As a final check for consistency, a check whether the same optimal result is obtained (step 72). If not, the customization has to be performed again.

If the final check for consistency is cleared, a duration of therapy as deemed appropriate by a qualified medical practitioner is selected (Step 74). Each therapy session may be 30 minutes lasting for a range of 6 weeks to 3 months.

The patient 100 is also given a choice of whether he wishes to proceed with the therapy (Step 76). The patient's data is saved for future use (Step 78) if he does not wish to proceed with the therapy.

As an example, for the patient 100 of FIG. 10 who decides to proceed with the therapy to improve his arterial pulse waveform, he would position the plunger 20 at the P₂ position with a tension of T₂ for a period of time as determined by a qualified medical practitioner.

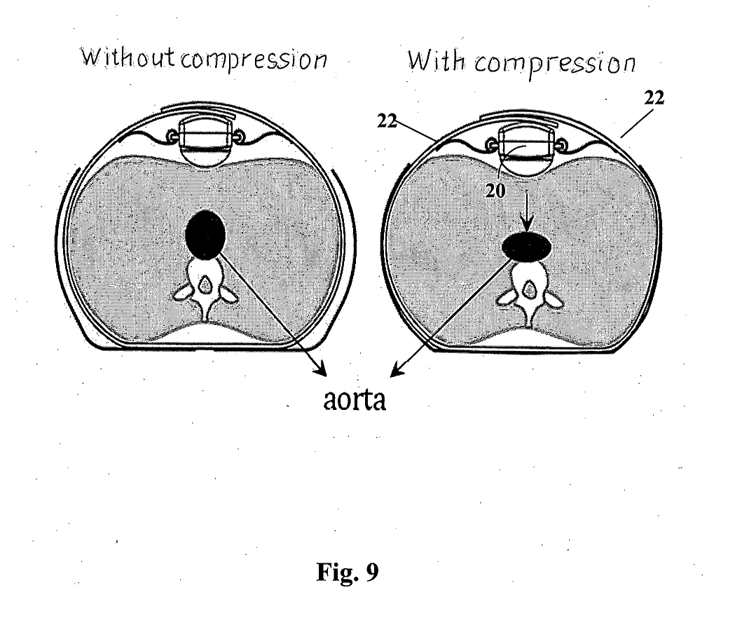

By compressing a pressure point P₁, P₂ and ensuring that overall circumference of the abdomen is not constricted, the apparatus 10 exerts only a downward pressure on the aorta 120 of the patient 100. The Applicant discovered that the downward pressure alters or slow down the return of the reflected wave during diastole phase (see FIG. 9).

The above described design removes any synchronization to time compression specifically before the onset of systole/diastole. No continuous ECG monitoring is required, as the customization stage is separate and independent from the treatment stage.

The above apparatus is especially suited for patients who do not qualify to go through by-pass; is a cheaper alternative to external/extracorporeal counter pulsation (EECP) life support with intra aortic balloon pump (IABP); and is easier and more convenient to use.

A summary of the advantages of the apparatus and method as compared to the enhanced external counter pulse (EECP) system is summarized as follows:

As compared to the (EECP) apparatus, the current invention does not require any form of synchronization with the patient's ECG cycle, thus eliminating the possibilities of such errors. In addition, there is no active ‘squeezing’ of the region (thighs and/or hip/buttocks) wrapped by cuffs which may cause bruises.

The present invention relies on analysis of pulse waveform instead of ECG as the main determinant. It is to be appreciated that pulse waveform is a more accurate measure as it is more closely associated with the mechanical movement of the heart and valves, as compared to ECG which may be subjected to errors arising from mechanical-electrical dissociation.

In addition, the present invention does not require active rhythmic or synchronized pulsation. It is thus envisaged that the invention, once appropriately customized, may be used an accessory while the patient 100 is doing his day-to-day routine and work.

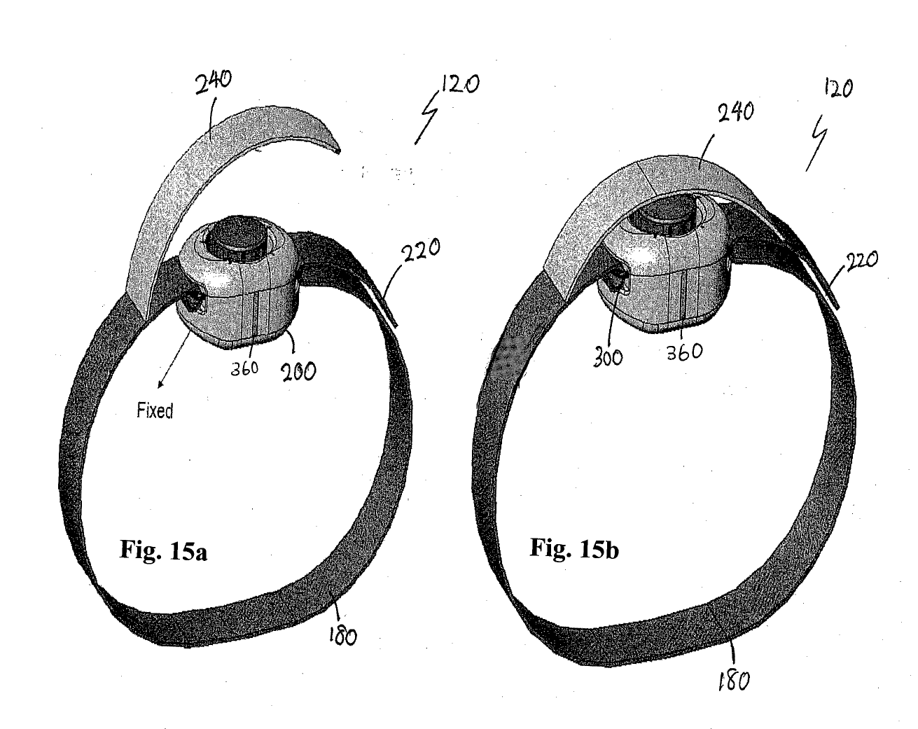

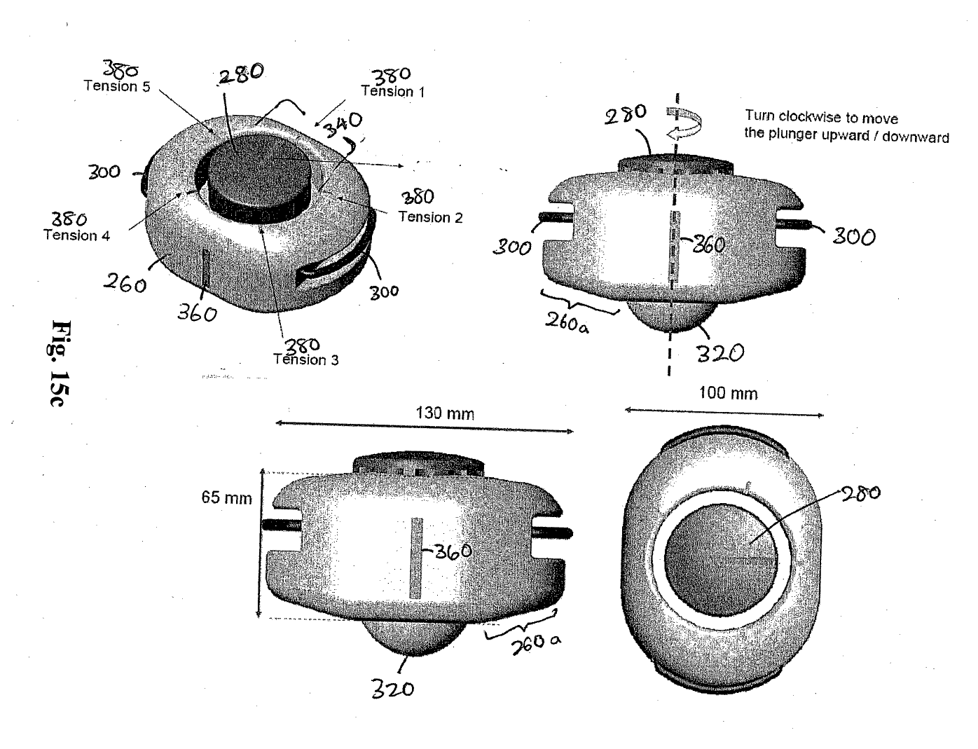

FIG. 15 illustrates an alternative compression belt 120 for use with the apparatus 10. Alternative compression belt 120 may be used to replace the compression belt 12 illustrated in FIG. 2.

The compression belt 120 comprises a waist strap 180, compression member 200, and an elastic band 240.

Waist strap 180 may be attached to the compression member 200 via securing handles 300. Waist strap 180 comprises fastening means 220 for adjusting and securing the compression belt 120 over the abdominal region of the body 100.

Fastening means 220 may be in the form of a Velcro™ surface as known to a person skilled in the art. Preferably, the width of the waist strap 180 is about a minimum of 55 millimetres to provide maximum comfort to the wearer.

Compression member 200 comprises a base member 260, a knob 280, securing handles 300 and plunger 320.

The base member 260 comprises a centre portion 340 adapted to receive the knob 280 and plunger 320. The knob 280 and plunger 320 are arranged such that turning the knob 280 in a clockwise or anti-clockwise direction protract or retract the plunger 320 with respect to the centre portion 340. Such an arrangement may be achieved for example via a screw assembly.

One end of the elastic band 240 is affixed to the waist strap 180 at a portion adjacent to one securing handle 300 of the compression member 200. The other end of the elastic band 240 may be removably attached to the waist strap 180 via for example a Velcro™ surface at a portion adjacent to the other securing handle 300. When secured on a body 100, the elastic band 240 covers the knob 280 and the compression member 200. The elastic band 240 assists in pressing the compression members against pressure points P₁ and P₂ as previously described and holding the compression member 200 against the pressure points. In the secured position, the elastic band 240 covers the knob 280 as shown in FIG. 15b.

The bottom surface of the base member 260, where the plunger 320 protracts or retracts from, comprises two inclinations 260a sloping towards the two opposite sides of the base member 260. In use, as the plunger 320 urges towards the pressure point P₁ or P₂ of the body 100, the inclinations 260a provides a form of adaptation to the shape of the abdominal region adjacent to the pressure point P₁ and/or P₂. As pressure is applied to the pressure point P₁ or P₂ via the plunger 320, the abdominal regions adjacent to the pressure points P₁ or P₂ curve towards and contacts the inclinations 260a and thereby forms a wrap around compression member 200. Such an arrangement restricts the compression member 200 from moving away from the pressure point P₁ or P₂. The two inclinations 260a are especially useful for people who are relatively fatter.

A marking 360 may be made on base member 260. Marking 360 corresponds to the point of the plunger 320 where maximum pressure is exerted against the abdominal region of the body 100 at each given pressure level T₁, T₂, T₃, T₄, and T₅. Further markings 380 may be made on the turning knob 280 and base member 260 to indicate the various pressure levels. Five pressure levels T₁, T₂, T₃, T₄, and T₅ are illustrated, but more of less pressure levels (at least two pressure levels) may be provided as known to a skilled person.

In use, the compression member 200 contacts a pressure point P₁ or P₂ on the abdominal region of the body 100 and exerts a pressure level T₁, T₂, T₃, T₄, or T₅ on the abdomen via turning the knob 280 clockwise or anti-clockwise to correspond to each defined marking 360.

It is appreciated that the alternative compression belt 120 as described does not require adjustment of the waist strap 180 to adjust the pressure levels, unlike the compression belt 12. Instead, the user adjusts the pressure tension/levels T₁, T₂, T₃, T₄, and T₅ applied to the body 100 via turning the knob 280 instead of adjusting the waist strap 180, which is more convenient and intuitive for the user. Further, the user does not have to concern himself with the proper fastening of the compression belt 120 at the different diameters in order to avoid constricting the overall circumference of the abdomen.

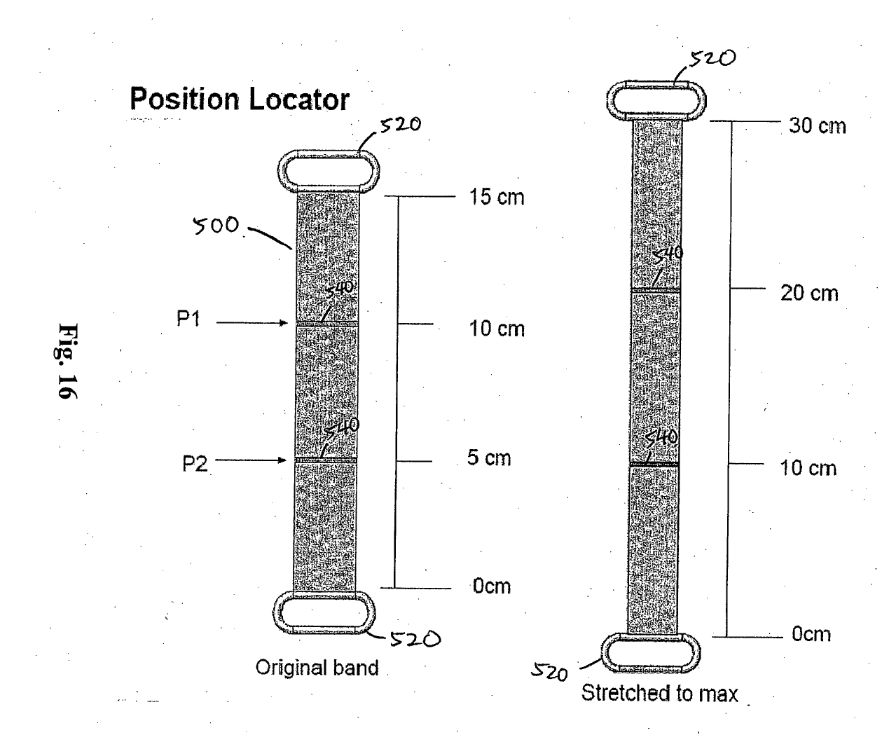

FIGS. 16a and 16b illustrate a position locator which may be used as an alternative to the marking method described in FIG. 5. The position locator comprises an elastic band 500 with two handles 520 affixed to each end of the elastic band 500. Markings 540 are made to the elastic band at the ⅓ and ⅔ distance with respect to one of the handles 520. For example, an elastic band 500 which is 15 centimeters (cm) long will have a marking 540 at 5 cm mark and another marking 540 at 10 cm mark.

In use, the elastic band 500 may replace steps 34, 36 and 38 as described. The elastic band 500 is simply stretched via holding the two handles 520 such that one end of the elastic band 500 is placed on the Xiphis sternum and the elastic band 500 is stretched so that the other end is on the navel of the patient 100. The markings 540 will locate the two pressure points P₁ and P₂ dividing the three segments marked out. This saves time in measuring the length between the xiphis sternum and navel for each different patient 100 and marking the distance between them.

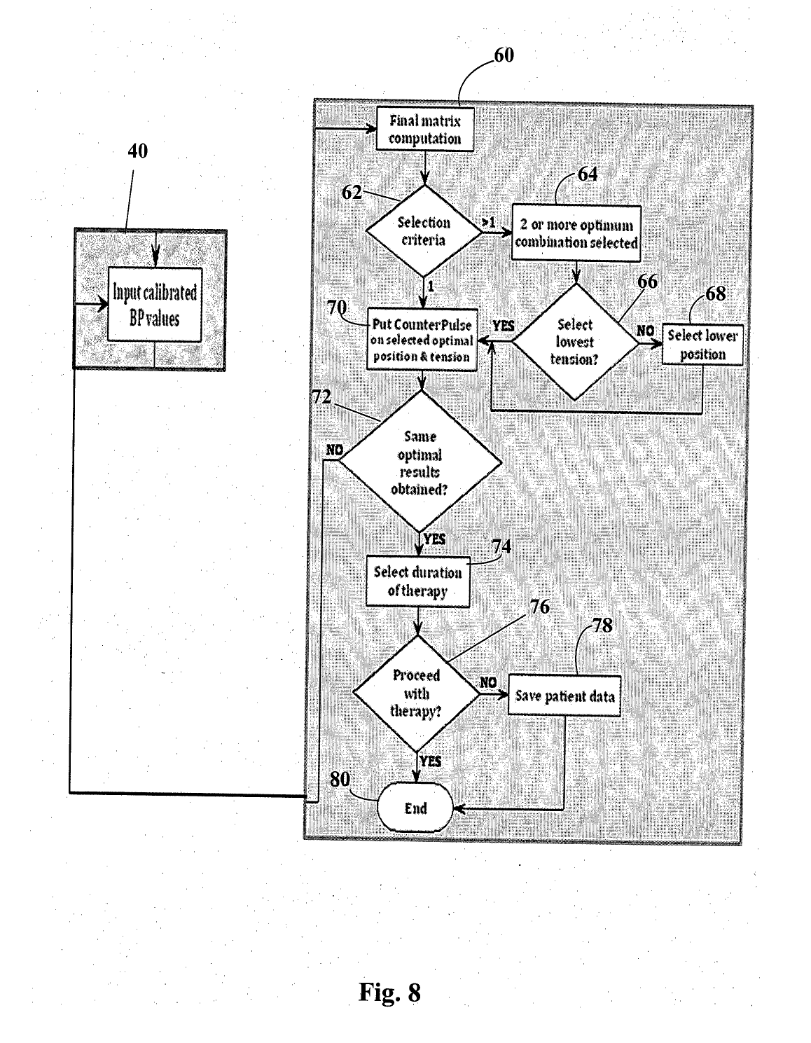

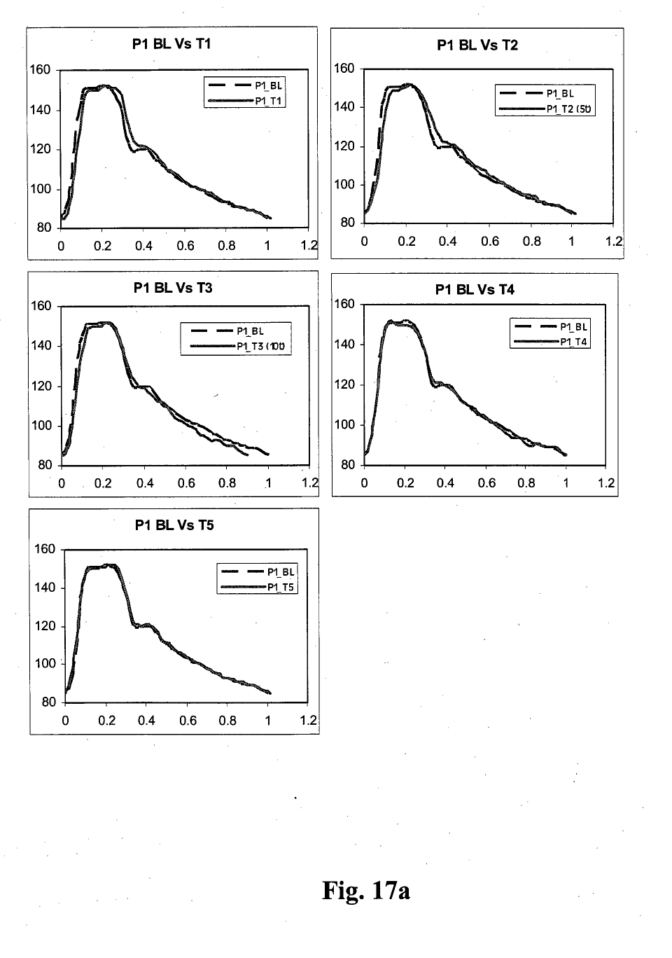

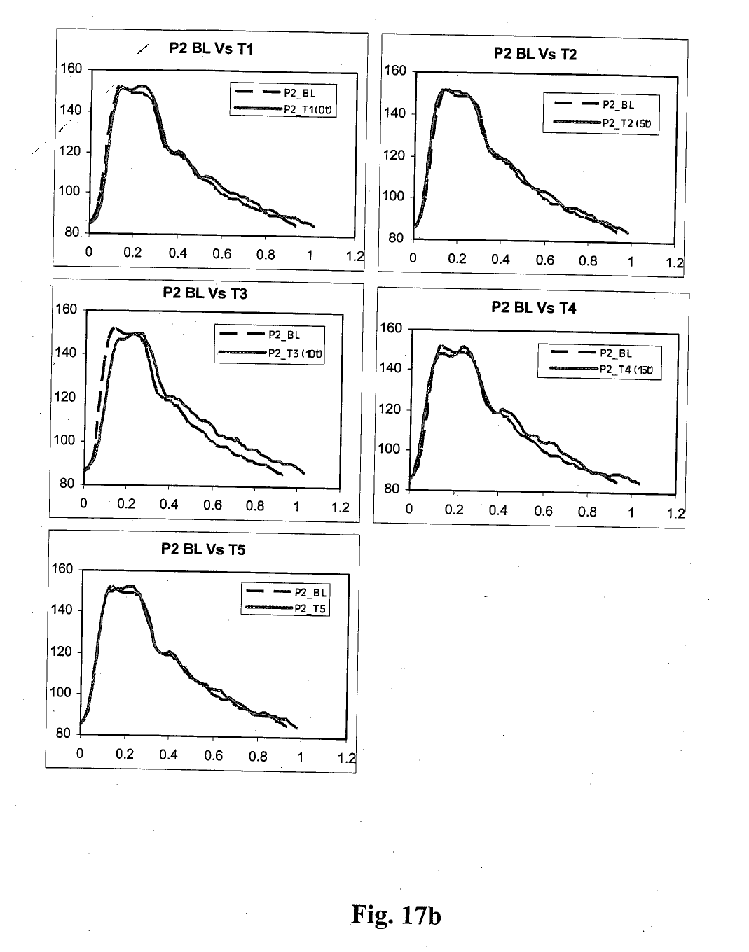

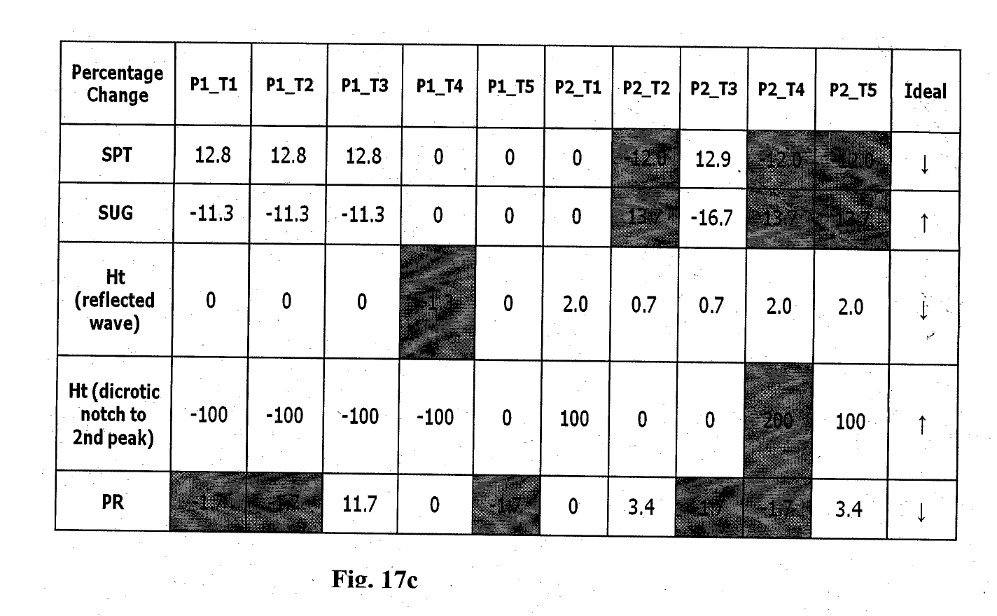

Further result based on the alternative compression belt 120 is shown in FIGS. 17a to 17c. FIG. 17c shows the presents each of the five indices corresponding to each combination of pressure points {P₁ or P₂} and tension {T₁; T₂; T₃; T₄; and T₅} in a matrix database of a particular patient 100 having a relatively unhealthy profile. FIG. 17c is obtained using similar steps described in FIG. 6 to FIG. 8, except that five tension levels are used instead of three.

As illustrated by FIG. 17c, the {P₂, T₄} combination provides the best improvements in four indices out of five, with improvements in SPT (12%), SUG (13.7%), net dicrotic notch to peak height (200%) and pulse rate (1.7%). The only index which does not improve is the reflected wave index but the overall benefits from the improvements in four indices far outweigh that of the reflected wave index.

It is also discovered that the use of the apparatus 10 using the alternative compression belt 120 will not significantly affect a patient 100 who is relatively healthy.

It should be appreciated by the person skilled in the art that the above invention is not limited to the embodiment described. In particular, the following modifications and improvements may be made without departing from the scope of the present invention:
Customization of the apparatus 10 according to the patient 100 profile may include more pressure points in addition to P₁ and P₂. Additional pressure points are selected from the distance XSN between the xiphis sternum and navel of the patient 100.
Each of the arterial pulse waveform index may be weighted (higher weightage for more important parameters). Preferably, the net dicrotic notch to peak height is given a heavier weightage as compared to other indices.
More or less waveform indices may be included/excluded to the five arterial waveform indices. For example, RNA (ratio of net area), rAI (radial augementation index), rAP (radial augmentation pressure), PRT (relative time between SBP and augmentation), MAP (mean arterial pressure)
A feedback system may be added to ensure that the optimal waveform of the patient is obtained at various time intervals after the treatment process has commenced. The feedback system may prompt for a change in pressure level/tension and/or pressure point according to the profile/needs of the patient 100.
Instead of using three or five pressure level/tension T₁, T₂, T₃, T₄, and T₅, a dual pressure level/tension system may be used. An example of the updated matrix according to a dual pressure level/tension is shown in FIG. 12.
While the radial arterial pulse waveform is described in the embodiment, arterial pulse waveforms obtained from other artery, for example brachial and carotid arteries, as known by a person skilled in the art may be obtained.
The selection of the optimal tension, pressure point combination could also be automated based on heuristic algorithms or optimization algorithm as known to a skilled person.

It is to be further appreciated that various aspects of the embodiments as described may be combined to form further embodiments within departing from the scope of the invention.

1. An apparatus for altering the arterial pulse waveform of a body comprising:
a belt having a compression member arranged to apply a pressure to the body, the belt being arranged to be positioned such that the compression member applies the pressure to one of at least two pressure points between the xiphis sternum and navel of the body, the belt adjustable to provide at least two pressure levels to each of the at least two pressure points and
a blood pressure monitoring device arranged to:
obtain a beat-to-beat arterial pulse waveform when the pressure is applied at each of the pressure points and at each of the at least two pressure levels; and
analyze the waveform to determine the optimum position for the compression device to apply the pressure to the body to alter the pulse waveform.

2. An apparatus of claim 1, wherein the beat-to-beat arterial pulse waveform is obtained from a radial artery.

3. An apparatus of claim 1 or 2, wherein the beat-to-beat blood pressure monitoring device is non-invasive.

4. An apparatus of claim 3, wherein the arterial pulse waveform analyzer is integrated with the beat-to-beat blood pressure monitoring device.

5. An apparatus of any of the preceding claims, wherein the at least two pressure points between the xiphis sternum and navel of the body are located at approximately at the ⅓ and ⅔ of the distance between the xiphis sternum and navel.

6. An apparatus of any of the preceding claims, wherein the applied pressure varies according to the adjustment of the belt to at least two diameters around the body.

7. An apparatus of claim 1, wherein the compression member comprise a knob and a plunger assembly adapted to adjust the pressure level applied to each of the at least two pressure points.

8. An apparatus of claim 7, wherein the compression member is adjustable to at least five different pressure levels.

9. An apparatus according to claim 8, wherein the compression member is shaped to adapt to the body curvature such that in use, a part of the body curves and form a partial wrap around the compression member to hold the compression member on the pressure point.

10. A method for altering the arterial pulse waveform of a body comprising the steps of:
a. locating the xiphis sternum and navel of the body;
b. determining the distance between the xiphis sternum and navel;
b. selecting a pressure point on the distance; and
d. applying pressure to the pressure point for a predetermined period.

11. The method of claim 10, wherein the applied pressure is substantially constant throughout the predetermined period.

12. The method of claim 10 or 11, wherein the pressure point is selected from at least two pressure points located on the distance between the xiphis sternum and navel.

13. The method of claim 12, wherein the at least two pressure points are located at approximately at the ⅓ and ⅔ of the distance between the xiphis sternum and navel.

14. The method of claim 13, wherein the pressure point and amount of pressure exerted is at the pressure point are determined based on the body's response to at least one arterial waveform indices.

15. The method of claim 14, wherein the arterial waveform indices include one or more of the following: Systolic peak time (SPT); Systolic upstroke gradient (SUG); Augmentation produced by reflected arterial wave; Net dicrotic notch to peak height; and Pulse rate (PR).

16. A computer readable medium having computer-executable instructions for performing the method recited in claims 10 to 15.
